(19) United States
(12) Patent Application Publication
(10) Pub. No.: US 2024/0065994 A1
(43) Pub. Date: Feb. 29, 2024

(54) COMPOSITIONS AND METHODS FOR PREVENTING OR TREATING DISEASES OR DISORDERS ASSOCIATED WITH PROTEIN MISFOLDING

(71) Applicant: Sailendra Nath Nichenametla, Croton-on-Hudson, NY (US)

(72) Inventor: Sailendra Nath Nichenametla, Croton-on-Hudson, NY (US)

(21) Appl. No.: 18/494,441

(22) Filed: Oct. 25, 2023

Related U.S. Application Data
(62) Division of application No. 17/349,808, filed on Jun. 16, 2021.
(60) Provisional application No. 63/039,808, filed on Jun. 16, 2020.

(51) Int. Cl. A61K 31/197; A61P 3/00; A61P 3/10; A61P 25/00; A61P 43/00

(52) U.S. Cl. CPC A61K 31/197; A61P 3/00; A61P 3/10; A61P 25/00; A61P 43/00

(57) ABSTRACT

Methods and uses are disclosed herein. A method of treating a disease or disorder associated with improper protein folding within one or more of a subject's cells may include administering an effective amount of a treatment compound into the subject. The treatment compound includes buthionine sulfoximine. The treatment compound provides improved redox conditions to enhance the unfolded protein response and the degradation of protein aggregation. The buthionine sulfoximine may decrease cellular glutathione to achieve the desired oxidative milieu, but not to the extent where cellular toxicity occurs.

### CROSS-REFERENCE TO RELATED APPLICATION

This application is a divisional application of U.S. patent application Ser. No. 17/349,808, filed Jun. 16, 2021, pending, which claims the benefit of U.S. Provisional Application No. 63/039,808 filed on Jun. 16, 2020, each of which is incorporated by reference in its entirety.

### TECHNICAL FIELD

The disclosure relates to compositions and methods of providing improved health benefits for subjects that have diseases or disorders associated with improper protein folding. More specifically, the disclosure relates to providing pharmaceutical compositions that to reduce protein misfolding and aggregation to support the proper function of certain organs of the body.

### BACKGROUND

Proteins are complex macromolecules essential for the proper functioning of cells and tissues in all living organisms. Proteins carry out numerous functions, including providing a structural framework for cytoskeleton, components of the membranes of subcellular organelles, acting as signal transducers, and catalyzing several biochemical reactions. The functional competency of each protein depends on its unique three-dimensional conformation, referred to as “native” structure. Cells have dedicated organelles and mechanisms to impart native structures to newly synthesized “nascent proteins,” as well as mechanisms to degrade misfolded proteins, or proteins that otherwise fail to attain native structure. A decrease in the efficiency of these systems results in the aggregation of misfolded proteins, which eventually become toxic to cells. Cells could also die due to the deprivation of essential functions that a misfolded protein would have performed if it were native, or an unfolded protein would have performed if it became native. Protein misfolding and aggregation plays a causal role in several diseases, including metabolic diseases such as diabetes, neurodegenerative diseases such as Alzheimer's diseases, amyotrophic lateral sclerosis, and hepatic steatosis, and the aging process in general. See Gregersen, N., et al. “Protein misfolding and human disease” Annu Rev Genomics Hum Genet, 7, 103-24, 2006; and Gruys, E. “Protein folding pathology in domestic animals” J Zhejiang Univ Sci, 5, 1226-38, 2004), the relevant parts of which are incorporated herein by reference. In one specific example, Mutant INS-gene-Induced Diabetes of Youth (MIDY), is characterized by an impairment in the folding of proinsulin due to genetic mutations.

These and other protein misfolding diseases and disorders are often managed by multiple daily injections or continuous subcutaneous infusion. The problem with these methods is that they can cause lipodystrophy at the injection site, ketoacidosis, hypoglycemia, vascular complications, increased mortality. Additionally, these management tools can be expensive when the subject has to purchase equipment such as insulin pumps, for example. Furthermore, exogenous therapies such as insulin therapy often do not accurately simulate blood glucose levels or homeostasis in the pancreas. Thus, therapeutic options are needed to boost the ability of cellular machinery to properly fold newly synthesized proteins and degrade aggregates of misfolded proteins generally and enhance the inherent ability of patients to control blood glucose levels specifically.

### SUMMARY

Embodiments of the present invention include pharmaceutical compositions and methods for the treatment of diseases or disorders associated with improper protein folding within one or more of a subject's eukaryotic cells. In one embodiment, the disease or disorder is diabetes. The pharmaceutical compositions may include an effective amount of buthionine sulfoximine and one or more pharmaceutically acceptable excipients. The reducing and oxidizing equivalents required for the reduction and oxidation of cysteine residues in the endoplasmic reticulum are provided by the interconversion of reduced glutathione (GSH) and oxidized glutathione or glutathione disulfide (GSSG). The efficiency by which the endoplasmic reticulum processes nascent proteins to native and functional proteins depends on its ability to maintain an oxidizing milieu by regulating the concentrations of GSH and GSSG. In one embodiment, the pharmaceutical composition is configured to reduce levels of glutathione within the endoplasmic reticulum of a subject's eukaryotic cells, thus creating optimal or improved oxidative milieu conditions for decreased protein misfolding. In another embodiment, the pharmaceutical composition is configured to create a redox environment within the cell that is conducive to increase the cell's unfolded protein response and increase the degradation of protein aggregation.

Other embodiments describe methods of treating diseases or disorder associated with improper protein folding within one or more of a subject's cell. In certain embodiments the diseases or disorders relate to diabetes. The method may include administering an effective amount of a pharmaceutical compound that includes buthionine sulfoximine to a subject to reduce instances of improper protein folding. Embodiments of the method may include administering an effective amount of a pharmaceutical compound that includes buthionine sulfoximine to a subject to reduce glucose levels in the subject's eukaryotic cells. Embodiments may also include the use of the pharmaceutical composition in the manufacture of a medicament for treating a disease or disorder associated with improperly folded proteins within a subject, including without limitation, diabetes.

Accordingly, embodiments of the present invention address unmet needs in the prior art by increasing the efficiency by which the endoplasmic reticulum processes nascent proteins to native and functional proteins by creating the proper oxidizing milieu in the cell.

### BRIEF DESCRIPTION OF THE DRAWINGS

FIG. 1 is a plot range graph comparing the weight of healthy and diabetic mice weight at various treatment times;

FIG. 2 is a bar graph comparing cumulative food intake between healthy and diabetic mice;

FIG. 3 is a bar graph comparing glutathione levels in healthy and diabetic mice;

FIG. 4 is line graph comparing blood glucose levels in healthy and diabetic mice at various treatment times;

FIG. 5 is a bar graph comparing area under the curve (AUC)-Baseline data for glucose tolerance in healthy and diabetic mice; and

FIG. 6 is a bar graph comparing cumulative area under the curve for insulin tolerance for healthy and diabetic mice.

### DETAILED DESCRIPTION

In the following detailed description, reference is made to the accompanying drawings which form a part hereof. The detailed description includes various embodiments of the compositions and methods of the present invention. These embodiments are described in sufficient detail to enable those of ordinary skill in the art to practice the disclosure. However, before the present materials and methods are described, it is to be understood that this invention is not limited to the particular molecules, compositions, active ingredients, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein. Accordingly, various substitutions, modifications, additions rearrangements, or combinations thereof are within the scope of this disclosure. Furthermore, all or a portion of any embodiment disclosed herein may be utilized with all or a portion of any other embodiment, unless stated otherwise.

The section headings provided herein are for convenience only do not interpret the scope or meaning of the claimed options. Furthermore, unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

### DEFINITIONS

As used herein and in the appended claims, the singular forms “a”, “an” and “the” include plural reference unless the context clearly dictates otherwise.

The terms “A or B,” “at least one of A and B,” “one or more of A and B”, or “A and/or B” as used herein include all possible combinations of items enumerated with them. For example, use of these terms, with A and B representing different items, means: (1) including at least one A; (2) including at least one B; or (3) including both at least one A and at least one B. In addition, the articles “a” and “an” as used herein should generally be construed to mean “one or more” unless specified otherwise or clear from the context to be directed to a singular form.

The expression “configured to” as used herein may be used interchangeably with “suitable for,” “having the capacity to,” “designed to,” “adapted to,” “made to,” or “capable of” according to a context. The term “configured” does not necessarily mean “specifically designed to,” and the expression compound or composition “configured to . . . ” may mean that the compound or composition is “capable of . . . ” along with other compounds or compositions in a certain context.

Unless the context otherwise requires, in the description text and in the claims that follow, the term “contain” and its derivatives, such as “contains” and “containing,” should be considered open, non-restrictive forms, that is, as “including but not limiting.” In addition, the terms “having,” or “including” should be understood as “including but not limited to the specific member or members listed. Thus, a compound or composition that “contains” or “includes” buthionine sulfoximine as an active ingredient may have additional active ingredients or compounds.

The term “about,” as used herein, includes any value that is within 10% of the described value.

The term “between,” as used herein, is inclusive of the lower and upper number of the range.

Reference herein to any numerical range (for example, a dosage range) expressly includes each numerical value (including fractional numbers and whole numbers) encompassed by that range. For example, but without limitation, reference herein to a range of 0.5 mg/dL to 100 mg/dL explicitly includes all whole numbers and fractional numbers between the two.

As used herein, the terms “administration” and “administering” refer to the act of providing a therapeutic, prophylactic, or other agent to a subject for the treatment or prevention of one or more diseases or disorder. Exemplary routes of administration to the human body are through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the term “disease or disorder” includes any and all conditions, symptoms, and/or effects that may be associated with the disease or disorder.

As used herein, the term “pharmaceutical composition” refers to an agent or active ingredient (e.g., BSO) with or without a carrier, excipient, or other ingredient or impurities, whether inert or active, making the composition suitable for diagnostic or therapeutic use in vitro, in vivo or ex vivo. The term “pharmaceutical composition” is meant to include any medicament, therapeutic, supplement, and the like.

The term “subject” refers to an animal, including, but not limited to, a primate (e.g., human), cow, pig, sheep, goat, horse, dog, cat, rabbit, rat, or mouse. The terms “subject” and “patient” are used herein interchangeably in reference, for example, to a mammalian subject, such as a human subject, in one embodiment, a human. In one embodiment, the subject has or is susceptible to having a disease or disorder provided herein.

The use of “diet” when used in conjunction with a subject may refer to the normal or typical diet, including without limitation, the caloric and/or nutritional consumption, of the particular subject. The “diet” of a subject may also refer to a recommended daily allowance of calories or nutrients for the particular age, height, and weight of a particular subject.

The terms “treat,” “treatment,” or “treating”, as used herein, refer to any method used to partially or completely alleviate, ameliorate, relieve, inhibit, prevent, delay onset of, reduce severity of and/or reduce incidence of one or more symptoms, conditions, or features of a disease or disorder. Treatment may be administered to a subject who does not exhibit signs of a disease or disorder. In some embodiments, treatment may be administered to a subject who exhibits only early signs of the disease or disorder for the purpose of decreasing the risk of developing pathology associated with the disease or disorder. It will be appreciated that, although not precluded, treating a disease or disorder does not require that the disease, disorder, or conditions or symptoms associated therewith be completely eliminated.

The terms “prevent,” “preventing” or “prevention,” as used herein, include inhibiting or preventing a disease or disorder and any conditions, symptoms, or effects thereof as well as preventing or inhibiting the underlying causes of such diseases, disorders, conditions, symptoms, e.g., arresting the development of the disease or disorder and are intended to include prophylaxis. The terms further include achieving a prophylactic benefit. For prophylactic benefit, the compositions are optionally administered to a patient at risk of developing a particular disease or disorder to a subject reporting one or more of the physiological symptoms of a disease, disorder, or condition or to a subject at risk of reoccurrence of the disease or disorder.

The terms “effective amount” or “therapeutically effective amount” as used herein, refer to a sufficient amount of at least one agent, compound, or compositions being administered which achieve a desired result, e.g., to relieve to some extent one or more symptoms of a disease or disorder being treated. In certain instances, the result is a reduction and/or alleviation of the signs, symptoms, or causes of a disease, or any other desired alteration of a biological system. In certain instances, an “effective amount” for therapeutic uses is the amount of the agent at dosages and for periods of time necessary, to achieve the desired therapeutic or prophylactic result. As will be apparent to those skilled in the art, it is to be expected that the effective amount of an agent, compound, or composition disclosed herein may vary according to factors such as the disease state, age, sex, and weight of the individual, and the ability of the treatment compound or composition to elicit a desired response in the particular individual. An “effective amount” or “therapeutically effective amount” is also one in which any toxic or detrimental effects of the treatment agent, compound, or composition are outweighed by the therapeutically beneficial effects.

The term “pharmaceutically acceptable” as used herein, refers to a material that does not abrogate the biological activity or properties of the agents, compounds, or compositions described herein, does not substantially produce adverse, allergic, or immunological reactions when administered to a subject, relatively nontoxic (i.e, the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

As used herein, “pharmaceutically acceptable salts” refer to derivatives of the treatment compound or composition wherein the treatment compound or composition is modified by reacting it with an acid or base as needed to form an ionically bound pair. Examples of pharmaceutically acceptable salts include conventional non-toxic salts or the quaternary ammonium salt of the parent compound formed, for example, from non-toxic inorganic or organic acids. Suitable non-toxic salts include those derived from inorganic acids such as hydrochloric, hydrobromic, sulfuric, sulfamic, phosphoric, nitric, and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, benzoic, salicylic, sulfanilic, fumaric, oxalic, isethionic, and others known to those of ordinarily skilled in the art. List of other suitable salts are found in Remington's Pharmaceutical Sciences, 17th edition. Mack Publishing Company, Easton Pa., 1985. p. 1418, the relevant disclosure of which is hereby incorporated by reference.

The term “carrier” as used herein, refers to relatively nontoxic chemical agents that, in certain instances, facilitate the incorporation of an agent into cells or tissues.

As used herein, “pharmaceutically acceptable carrier” includes any material which, when combined with a compound or composition of the invention, allows the compound or composition to retain biological activity, such as the ability to treat the associated disease or affect the various mechanisms associated therewith, and is non-reactive with the subject's immune system. Examples include, but are not limited to, any of the standard pharmaceutical carriers such as a phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers, and adjuvants, see, e.g., Martin, Remington's Pharmaceutical Sciences, 15th Ed., Mack Publ. Co., Easton, Pa. (1975), incorporated herein by reference in its entirety.

“Pharmaceutically acceptable excipients,” as used herein, include but are not limited to binders, diluents, lubricants, disintegrants, glidants and surface-active agents. The amount of excipient employed will depend upon how much active agent is to be used. One excipient can perform more than one function.

The term “improper protein folding,” includes protein misfolding, proteins that don't fold and/or remain unfolded, and protein aggregation. Similarly, the term “improperly folded protein(s),” includes proteins that are misfolded, unfolded, aggregating, or otherwise unable to perform their proper function. By way of nonlimiting example, the phrase “improper proinsulin folding is reduced or ameliorated” could mean that instances of proinsulin misfolding have been reduced, instances of unfolded proinsulin have been reduced, and/or instance of proinsulin aggregation have been reduced.

The term “protein misfolding” as used herein describes a process where a protein chain fails to acquire its native three-dimensional structure, where a protein chain does not fully or completely form or conformation from a polypeptide such that it doesn't perform its proper biological function, where a protein chain fails to be fully or properly translated from a sequence of mRNA into a linear chain of amino acids, where the protein folding process results in an inactive protein, where the amino acids of a protein become disordered, or where amino acid residues fail to properly interact such that the final structure of the protein is incorrect or in some way deficient.

The term “unfolded protein” as used herein describes a protein that doesn't fold, that is the result of protein denaturation, that transitions from a folded protein to an unfolded state, or where a protein is intrinsically disordered.

References to “high levels of glucose” means blood sugar levels that are higher than normal as determined by A1C tests, Fasting Blood Sugar tests, Glucose Tolerance tests, Random Blood Sugar tests, Glucose Screening tests, or other tests used to measure blood glucose levels.

The term “protein aggregation” means the aggregation of any misfolded or unfolded proteins. The term “protein aggregation” includes the result of a cell failing to fully or properly assist the protein in re-folding and the result of a cell failing to fully or properly degrade the unfolded protein. The “protein aggregation” includes exposed hydrophobic portions of the protein interact with the exposed hydrophobic patches of other proteins. “Protein aggregation” includes the formation of amorphous aggregates, oligomers, and amyloid fibrils.

As used herein, the term “diabetes” refers to the set of diseases and conditions known collectively as “diabetes mellitus,” including “type 1 diabetes,” “type 2 diabetes,” “gestational diabetes” (during pregnancy), “Mutant INS-gene-induced Diabetes of Youth” (MIDY), and other states that cause hyperglycemia. The term includes disorders in which the pancreas produces and/or secretes insufficient amounts of active/properly-folded insulin, and/or in which the cells of the body fail to respond appropriately to insulin (e.g., “insulin resistance”) thus preventing cells from absorbing glucose. As a result of the different, untreated forms of diabetes, glucose builds up in the blood. “Mutant INS-gene-induced Diabetes of Youth” (“MIDY”) is associated with insulin deficiency initiated by an attack of misfolded mutant proinsulin on bystander wild-type proinsulin in the endoplasmic reticulum.

As used herein, the term “wild-type,” refers to a gene or gene product (e.g., protein) that has the characteristics (e.g., sequence) of that gene or gene product isolated from a naturally occurring source and is most frequently observed in a population. In contrast, the term “mutant” refers to a gene or gene product that displays modifications in sequence when compared to the wild-type gene or gene product. It is noted that “naturally-occurring mutants” are genes or gene products that occur in nature but have altered sequences when compared to the wild-type gene or gene product; they are not the most commonly occurring sequence. “Synthetic mutants” are genes or gene products that have altered sequences when compared to the wild-type gene or gene product and do not occur in nature. Mutant genes orgene products may be naturally occurring sequences that are present in nature, but not the most common variant of the gene or gene product, or “synthetic,” produced by human or experimental intervention.

The term “buthionine sulfoximine,” which may be used interchangeably with the letters “BSO,” refers to any form or source of buthionine sulfoximine, including without limitation, analogs, derivatives, isomers, and salts of buthionine sulfoximine.

The term “glutathione” as used herein through is glutathione in its reduced state and may be used interchangeably with the letters “GSH.” The term “glutathione” as used here is to be distinguished from “oxidized glutathione”, which may be referenced with the letters “GSSG.”

### Protein Folding

The endoplasmic reticulum constitutes the starting point of the secretory pathway where secretory and membrane proteins are synthesized. Correct folding of these endoplasmic reticulum client proteins is required for their proper function, which may depend on different endoplasmic reticulum-resident determinants such as chaperones and folding catalysts. Imbalances between the burden of protein synthesis in the endoplasmic reticulum and the capacity of its folding machinery to activate the unfolded protein response (UPR), an adaptive cellular program, which is known by those of skill in the art to be conserved from yeast to human. The unfolded protein response (UPR) operates in all eukaryotic cells to adjust the protein folding capacity of the endoplasmic reticulum (ER) according to need. Environmental or physiological demands can lead to an imbalance between the protein folding load and the protein folding capacity in the endoplasmic reticulum lumen, resulting in an accumulation of unfolded or misfolded proteins, a condition termed “ER stress.” When unmitigated, endoplasmic reticulum stress due to improperly folded proteins can be toxic to cells and may trigger cell death.

Protein balance or protein homeostasis in the endoplasmic reticulum is connected to the formation of native disulfide bonds during client protein folding, which also requires reduction of non-native bonds. Endoplasmic oxidoreductin 1 (ERO-1) enzymes, among other pathways, generate disulfide bonds by reducing molecular oxygen. This oxidative mechanism may be antagonized by the low molecular weight thiol compound glutathione (GSH), which maintains a reduced fraction of PDIs. GSH-mediated reduction results in the formation of its dimeric oxidized form glutathione disulfide (GSSG). The presence of oxidizing and reducing components in the endoplasmic reticulum allows a dynamic control of the redox state, sometimes referred to herein as the oxidative milieu of the cell. The status of the glutathione redox couple (GSH-GSSG) is an accepted indicator of intracellular redox conditions. Accordingly, although reduction of glutathione may increase cellular oxidative stress, and therefore not a mechanism typically used for combating protein misfolding, embodiments of the present invention demonstrate that increasing cellular oxidative stress to a certain limited degree creates an oxidative milieu that may prevent improper protein folding.

In vivo studies in F344 rats demonstrate that a decrease in hepatic GSH is associated with an increase in both PDI and endoplasmic reticulum-1α, suggesting that the expression of these two proteins can be modulated by altering the GSH concentrations, (See Nichenametla, S. N., et al. “Sulfur amino acid restriction-induced changes in redox-sensitive proteins are associated with slow protein synthesis rates” Ann N Y Acad Sci, 1418, 80-94, 2018, appropriate portions of which are incorporated herein by reference). While the mutant forms have a much higher propensity, misfolding can also occur in wild type proinsulin, particularly under conditions that perturb endoplasmic reticulum folding environment. Proinsulin misfolding is considered as an early event in the etiology of type-2 diabetes. (See Arunagiri, A., et al. “Proinsulin misfolding is an early event in the progression to type 2 diabetes” Elife, 8, 2019, appropriate portions of which are incorporated herein by reference). In vitro studies in murine HT22 cells demonstrate that GSH depletion activates PDI by increasing its nitrosylation, suggesting that protein disulfide isomerase mediates glutathione depletion-induced cytotoxicity. (Okada, K., et al. “Protein disulfide isomerase mediates glutathione depletion-induced cytotoxicity,” Biochem Biophys Res Commun, 477, 495-502, 2016, appropriate portions of which are incorporated herein by reference).

Accordingly, maintaining optimal oxidative milieu through the reduction of GSH diminishes the misfolding of proteins, the number of unfolded proteins, and the amount of protein aggregation in a cell. Accordingly, compositions and methods that achieve or facilitate these conditions can be used to treat diseases or disorders associated with improper protein folding. When the protein is insulin, using the compositions and methods of the described embodiments to regulate such cellular conditions can diminish the negative effects of mutant proinsulin and retard progression of diabetes caused by misfolded proinsulin.

The experimental results discussed below show that effective amounts of BSO can reduce glucose levels in subjects and increase glucose tolerance in subjects without creating cellular toxicity and without significantly affecting insulin sensitivity. In one embodiment, effective amounts of BSO maintain optimal oxidative milieu through the reduction of GSH. Accordingly, embodiments of the present invention may reduce improper proinsulin folding specifically, and improper protein folding generally.

### Pharmaceutical Compositions

Embodiments of the disclosure describe compositions and methods for preventing or treating diseases associated with improper protein folding, including improper proinsulin folding which can lead to high levels of glucose in the blood and associated diseases and disorders such as diabetes, obesity, fatty liver, and the like. Embodiments of the disclosure are further directed to compositions and methods for treating diseases and disorders associated with high levels of glucose in a subject. Yet other embodiments of the disclosure are directed to methods and compositions for depleting cellular glutathione. In certain embodiments, these methods and compositions utilize buthionine sulfoximine as an agent or active ingredient. Accordingly, embodiments of methods and compositions of the present invention are useful in treating or preventing a number of diseases associated with improper protein folding generally and high levels of glucose specifically.

In one embodiment, a pharmaceutical composition for the prevention and treatment of diseases and disorders associated with improper protein folding in a subject's eukaryotic cells includes an effective amount of buthionine-sulfoximine (BSO). In one embodiment pharmaceutical composition includes an effective amount of buthionine sulfoximine having the molecular formula C₈H₁₈N₂O₃S. In one embodiment, the buthionine sulfoximine is L-buthionine-S-sulfoximine. In another embodiment, the buthionine sulfoximine is L-buthionine-R-sulfoximine. In yet another embodiment, the buthionine sulfoximine is D-buthionine-S-sulfoximine. In yet another embodiment, the buthionine sulfoximine is D-buthionine-R-sulfoximine. The BSO may also be pharmaceutically acceptable salts of the foregoing and/or combinations thereof. It will be appreciated by those of skill in the art that sources of BSO, may include, without limitation, BSO derivatives, BSO analogs, and other BSO isomers.

In one embodiment the BSO includes diastereomeric mixtures of composition L-buthionine-(S, R)-sulfoximine, having the chemical structure:

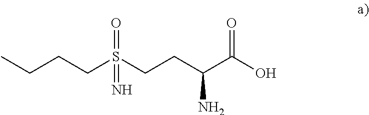

and composition D-Buthionine-(S, R)-sulfoximine, having the chemical structure:

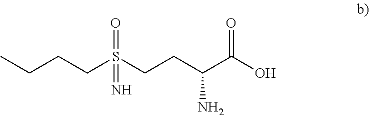

The diastereomers of a) and b) can be used as a mixture, or in their pure S and R isomer forms. In one embodiment, the effective amount of BSO is at least about 0.01 g/Kg body weight of the subject. The effective amount of BSO may also be between about 0.1 g/Kg body weight and about 10 g/Kg body weight of the subject. In yet other embodiments, the effective amount of BSO is between about 1 mg/Kg body weight and about 5 g/Kg body weight of the subject. In certain embodiments, the effective amount of BSO in the pharmaceutical composition is an amount sufficient to increase an oxidation level within a subject's eukaryotic cells without causing the cell to become toxic, when the pharmaceutical composition is administered to the subject. It will be appreciated by those of skill in the art that the Therapeutic Index or Therapeutic Ratio may be used to determine that amount of BSO that will cause a therapeutic effect and not cellular toxicity. In one embodiment, the effective amount of BSO falls within a therapeutic or safety window, which is a range of does with optimize between the efficacy of the pharmaceutical composition and the toxicity.

The effective amount of BSO may also be an amount sufficient to reduce a glutathione amount in the subject's eukaryotic cells. In one embodiment, the effective amount of BSO in the pharmaceutical composition may reduce a level or amount of glutathione in the subject's eukaryotic cells by at least about 1% when administered. In other embodiments, the effective amount of BSO may be such that when administered to a subject, the reduction of glutathione in the subject's eukaryotic cells is less than about 70%, 60%, 50%, 45%, 40%, 35%, 30%, 25%, 20%, 15%, 10%, or 5%. In one embodiment, the pharmaceutical composition containing an effective amount of BSO decreases an amount glutathione in the subject's eukaryotic cells, when administered to the subject, by an amount sufficient to increase an oxidation level within the subject's eukaryotic cells without causing the cells to become toxic.

In one embodiment, the effective amount of BSO, when administered to a subject, decreases cellular glutathione by decreasing its de novo biosynthesis. In another embodiment, the decrease in cellular glutathione may occur if BSO decreases the salvage of glutathione, or in other words, the cell's ability to pair up random parts into glutathione. It will be appreciated by those of skill in the art that salvaging is a pathway for creating glutathione. In other embodiments, the BSO may cause cellular depletion of glutathione by increasing its cellular export. In yet other embodiments, the reduction or depletion of glutathione may be caused by effective amounts of BSO occurs because there could be an increase in glutathione cleavage into constituent amino acids. In certain embodiments, an effective amount of BSO may cause a decrease in cellular glutathione through a combination of these depletion mechanisms.

It is well known in the art that glutathione (GSH) functions to protect cells by neutralizing reactive oxygen species becoming oxidized glutathione (GSSG) in the process. Thus, the ratio of glutathione to oxidized glutathione within cells is a measure of cellular oxidative stress. Decreasing glutathione increases the GSSG-to-GSH ratio, indicating greater oxidative stress. Additionally, in vivo GSH-to-GSSG ratios can be measured with subcellular accuracy using redox sensors to ensure that oxidative stress is not increased to the point of cellular toxicity.

In present embodiments, an effective amount of BSO has been determined that will increase oxidation levels in the subject's eukaryotic cell to a point where the cellular oxidative milieu creates a more effective environment for decreasing improper protein folding. Further, the effective amount of BSO does not increase oxidative stress to the point where cellular toxicity occurs.

Although an understanding of the mechanism is not necessary to practice the present invention and the present invention is not limited to any particular mechanism of action, in some embodiments, treatment with compositions and methods of the present invention may increase the unfolded protein response mechanism within the subject's eukaryotic cells. Further, the pharmaceutical composition may be configured with a sufficient amount of BSO to cause an increase in the degradation of protein aggregation in the subject's eukaryotic cells. Accordingly, embodiments of pharmaceutical compositions containing effective amounts of BSO may cause at least one of a reduced level of misfolded protein cells, a reduced level of unfolded protein cells, a reduced level of protein aggregation or combinations thereof. Because of BSO's effect on proinsulin misfolding, embodiments of the pharmaceutical compositions containing effective amounts of BSO may be used for the prevention and treatment of diseases and disorders associated with high levels of glucose in a subject's blood. In one embodiment, a pharmaceutical composition includes an effective amount of buthionine-sulfoximine. The source or makeup of buthionine-sulfoximine in this embodiment may be the same as discussed in other embodiments. Additionally, the effective amount of BSO in this embodiment may be the same as in other embodiments. In other words, a pharmaceutical composition may have an amount of BSO such that when administered to a subject, there is a decrease in glutathione levels. Thus, the effective amount of BSO may be one that regulates oxidative milieu in a eukaryotic cell such the improper proinsulin folding is reduced or ameliorated. In one embodiment, an effective amount of BSO may increase an oxidation level in the eukaryotic cell without causing the cell to become toxic. Further, the effective amount of BSO in this embodiment, may increase the unfolded protein response for proinsulin and decrease protein aggregation of unfolded or misfolded proinsulin.

In one embodiment, the pharmaceutical composition with an effective amount of BSO, decreases glutathione levels in the subject's eukaryotic cells to increase a cellular oxidation level without significantly increasing proinsulin sensitivity. In some embodiments, the pharmaceutical composition may reduce the subject's blood glucose levels by more than about 3%. In other embodiments, the pharmaceutical composition may reduce the subject's blood glucose levels by more than about 6%. In yet other embodiments, the pharmaceutical composition may reduce the subject's blood glucose levels by more than about 9%. In yet other embodiments, the pharmaceutical composition may reduce the subject's blood glucose levels by more than about 12%. In yet other embodiments, the pharmaceutical composition may reduce the subject's blood glucose levels by more than about 15%. In yet other embodiments, the pharmaceutical composition may reduce the subject's blood glucose levels by more than about 18%. In yet other embodiments, the pharmaceutical composition may reduce the subject's blood glucose levels by more than about 21%. In yet other embodiments, the pharmaceutical composition may reduce the subject's blood glucose levels by more than about 24%. In yet other embodiments, the pharmaceutical composition may reduce the subject's blood glucose levels by more than about 27%. In yet other embodiments, the pharmaceutical composition may reduce the subject's blood glucose levels by more than about 30%.

In other embodiments, the pharmaceutical composition, with an effective amount of BSO, may increase glucose tolerance by more than about 1%. In other embodiments, the pharmaceutical composition may increase glucose tolerance by more than about 6%. In yet other embodiments, the pharmaceutical composition may increase glucose tolerance by more than about 5%. In yet other embodiments, the pharmaceutical composition may increase glucose tolerance by more than about 10%. In yet other embodiments, the pharmaceutical composition may increase glucose tolerance by more than about 15%. In yet other embodiments, the pharmaceutical composition may increase glucose tolerance by more than about 20%. In yet other embodiments, the pharmaceutical composition may increase glucose tolerance by more than about 25%. In yet other embodiments, the pharmaceutical composition may increase glucose tolerance by more than about 30%. In yet other embodiments, the pharmaceutical composition may increase glucose tolerance by more than about 35%. In yet other embodiments, the pharmaceutical composition may increase glucose tolerance by more than about 40%. In yet other embodiments, the pharmaceutical composition may increase glucose tolerance by more than about 45%. In yet other embodiments, the pharmaceutical composition may increase glucose tolerance by more than about 50%.

As discussed above, pharmaceutical composition embodiments of the present invention may include an active ingredient, which may be BSO. In certain embodiments the pharmaceutical composition is substantially just the active ingredient. In other embodiments the pharmaceutical composition may include pharmaceutically acceptable excipients, such as, without limitation, binders, diluents, lubricants, disintegrants, glidants, surface-active agents and/or combinations thereof.

In one embodiment, the pharmaceutical composition may include one or more binders including without limitation, starches such as potato starch, wheat starch, corn starch; microcrystalline cellulose; celluloses such as hydroxypropyl cellulose, hydroxyethyl cellulose, hydroxypropyl methyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other binders known to one of ordinary skill in the art and mixtures thereof.

The pharmaceutical composition may include one or more fillers or diluents, including without limitation, confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and other fillers or diluents known to one of ordinary skill in the art and mixtures thereof.

The pharmaceutical composition may also include one or more lubricants or glidant. The lubricants may be selected from, but not limited to those conventionally known in the art, such as Mg, Al or Ca or Zn stearate, polyethylene glycol, glyceryl behenate, mineral oil, sodium stearyl fumarate, stearic acid, hydrogenated vegetable oil and talc. The glidants may include, without limitation, silicon dioxide, magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

In one embodiment, the pharmaceutical composition may include one or more disintegrants including but not limited to starches, clays, celluloses, alginates, gums; cross-linked polymers, e.g, cross-linked polyvinyl pyrrolidone or crospovidone, cross-linked sodium carboxymethylcellulose or croscarmellose sodium, and cross-linked calcium carboxymethylcellulose, soy polysaccharides, and guar gum. Use of disintegrant according to the present invention may facilitate in the release of the BSO from pharmaceutical composition in the latter stage of delivering and assist in completely releasing the BSO in the proper dosage form.

The embodiments of the pharmaceutical compositions of the present invention may optionally contain a surface-active agent. The preferred agent may be copolymers composed of a central hydrophobic chain of polyoxypropylene (poly (propylene oxide)) and polyoxyethylene (poly (ethylene oxide)) that are well known as poloxamer. However, other agents may also be employed such as dioctyl sodium sulfosuccinate (DSS), triethanolamine, sodium lauryl sulphate (SLS), polyoxyethylene sorbitan and poloxalkol derivatives, quaternary ammonium salts or other pharmaceutically acceptable surface-active agents known to one ordinary skilled in the art.

The pharmaceutical dosage forms of embodiments of the invention may have an extended release coating. This coating helps pharmaceutical composition to release active ingredients such as BSO at the right time and for the required length of time. The extended release coating may include a hydrophilic or hydrophobic substance or combinations thereof.

The pharmaceutical composition embodiments of the present invention may also include without limitation, pharmaceutically acceptable carriers, preservatives, vehicles, and/or stabilizers. The pharmaceutical composition embodiments of the present invention may be combined with a pharmaceutically acceptable buffer, and the pH adjusted to provide acceptable stability, and a pH acceptable for administration.

It will be appreciated by those of skill in the art that certain excipients, carriers, preservatives, stabilizers, and/or buffers can perform more than one function and that the amount of these will depend upon how much BSO is to be used.

### Methods of Treatment or Prevention

Embodiments of the present invention also include methods of preventing or treating diseases and disorders associated with improper protein folding. The method may include administering an effective amount of buthionine sulfoximine into a subject to decrease improper protein misfolding in the subject's eukaryotic cells. The buthionine sulfoximine in this embodiment may have the same structure and effect on eukaryotic cells as described herein throughout. The step of administering an effective amount of BSO includes any and all of the effective amounts of BSO described herein and their effects and results. In one embodiment, the effective amount of BSO is at least about 0.01 g/Kg body weight of the subject. The effective amount of BSO may also be between about 0.1 g/Kg body weight and about 10 g/Kg body weight of the subject. In yet other embodiments, the effective amount of BSO is between about 1 mg/Kg body weight and about 5 g/Kg body weight of the subject. In another embodiment, the effective amount of buthionine-sulfoximine is an amount sufficient to increase an oxidation level within a eukaryotic cell without causing the cell to become toxic. Thus, the step of administrating a pharmaceutical composition with an effective amount of BSO may include using the therapeutic index to determine an efficacious amount that doesn't cause cellular toxicity.

Additionally, the method step of administering an effective amount of BSO into a subject, includes the individual steps required to cause all of the effects that BSO may have on a eukaryotic cell as described herein. Indeed, in some embodiments, the step of administering an effective amount of buthionine-sulfoximine to a subject includes reducing or depleting a level of glutathione in the subject's eukaryotic cells by less than about 50%, 45%, 40%, 35%, 30%, 25%, 20%, 15%, 10%, or 5%. The administration step may further include decreasing an amount or level of glutathione in the subject's eukaryotic cells by an amount sufficient to increase an oxidation level within the subject's eukaryotic cells without causing the cells to become toxic. The administration step may further include decreasing cellular glutathione by decreasing its de novo biosynthesis, decreasing the salvage of glutathione, increasing its cellular export and/or increasing glutathione cleavage into constituent amino acids.

The administrative step in this embodiment may include increasing the unfolded protein response (UPR) mechanism in eukaryotic cells by at least 5%. The administrative step may also include increasing the degradation of protein aggregation in eukaryotic cells by at least 5%.

Embodiments of the present invention also include a method of preventing or treating diseases and disorders associated with high levels of glucose. In this embodiment, the method may include administering an effective amount of an active ingredient into a subject to decrease levels of glutathione in the subject's eukaryotic cells. The active ingredient may decrease levels of glutathione in eukaryotic cells by decreasing glutathione biosynthesis de novo. The administration of active ingredient may decrease glutathione in eukaryotic cells by less than 50%, 45%, 40%, 35%, 30%, 25%, 20%, 15%, 10%, or 5%.

In one embodiment, the method of preventing or treating diseases and disorders associated with high levels of glucose includes administering BSO as the active ingredient to reduce glucose levels in the subject's eukaryotic cells, including pancreatic cells. An effective amount of BSO, as described herein throughout, may reduce glucose levels by one or more of the mechanisms described in conjunction with other embodiments herein. These may include, without limitation, reducing a level of glutathione within the subject's pancreatic cells, increasing a level of protein disulfide isomerase within the subject's pancreatic cells, and increasing a level of endoplasmic oxidoreductin within the subject's pancreatic cells. The amounts such reduction and increases are the same or similar to those discussed in conjunction with other embodiments herein. In one embodiment, the protein disulfide isomerase is PDI-1 of the family of PDIs and the endoplasmic oxidoreductin may be one or both of ERO-1α and ERO-1β. Furthermore, adding an effective amount of BSO in this method may increase the unfolded protein response for proinsulin and decrease protein aggregation of unfolded or misfolded proinsulin.

In one embodiment, administering an effective amount of the active ingredient reduces the subject's blood glucose levels by more than about 3%, 6%, 9%, 12%, 15%, 18%, 21%, 24%, 27%, or 30%. Administering an effective amount of the active ingredient may also increase glucose tolerance by more than about 1%, 5%, 10%, 15%, 20%, 25%, 30%, 35%, 40%, 45%, or 50%. The effective amount of BSO as an active ingredient in order to help reduce glucose levels is at least about 10%. In one embodiment, an effective amount of BSO to reduce glucose levels is an amount sufficient to increase an oxidation level within a pancreatic cell without causing the cell to become toxic.

For all method embodiments described herein, the step of administering the active agent or pharmaceutical composition, either of which may include BSO, into the subject may at least one of ingesting the active ingredient, injecting the active ingredient, applying the active ingredient topically or by other ways known in the art. These additional ways may include without limitation, orally, subcutaneously, intravenously, intranasally, intraopticaly, transdermally, topically, intraperitoneally, intramuscularly, intrapulmonary, vaginally, parenterally, rectally, or intraocularly. In order to administer the pharmaceutical composition in one or more of these ways, the pharmaceutical composition may be formulated in certain embodiments as oral dosage forms (e.g., tablets, capsules, gels), inhalations, nasal sprays, patches, absorbing gels, liquids, liquid tannates, suppositories, injections, I.V. drips, gels, salves, lotions, creams, other delivery methods, combinations of these, and the like.

### Formation

In certain embodiments, the active ingredient and/or pharmaceutical composition are put into dosage forms before administration. Examples of solid dosage forms include but are not limited to discrete units in capsules or tablets, as a powder or granule, or present in a tablet conventionally formed by compression molding. Such compressed tablets may be prepared by compressing in a suitable machine the three or more agents and a pharmaceutically acceptable carrier. The molded tablets can be optionally coated or scored, having indicia inscribed thereon and can be so formulated as to cause immediate, substantially immediate, slow, or controlled release of the hydrocodone and/or the acetaminophen. Furthermore, dosage forms of the invention can comprise acceptable carriers or salts known in the art, such as those described in the Handbook of Pharmaceutical Excipients, American Pharmaceutical Association (1986), relevant portions incorporated by reference herein.

The pharmaceutical composition of the invention can be formed by various methods known in the art such as by dry granulation, wet granulation, melt granulation, direct compression, double compression, extrusion spherization, layering and the like. The solvent or solvents used in wet granulation formation embodiments include all the solvents well known in the art or their mixtures thereof.

To prepare the present compositions into dosage forms for administration into a subject as described above, the active ingredients can be mixed with a suitable pharmaceutically acceptable carrier. Upon mixing of the compounds, the resulting composition can be a solid, a half-solid, a solution, suspension, or an emulsion. BSO can be used in any form. In some embodiments, targeted delivery of BSO can be directed to a particular tissue, including pancreas. Such compositions can be prepared according to methods known to those skilled in the art. The forms of the resulting compositions can depend upon a variety of factors, including the intended mode of administration and the solubility of the compounds in the selected carrier or vehicle.

In one embodiment, the active ingredients are mixed with a pharmaceutical excipient to form a solid preformulation composition comprising a homogeneous mixture of active ingredients of the present invention. When referring to these compositions as “homogeneous”, it is meant that the agents or active ingredients are dispersed evenly throughout the composition so that the composition can be subdivided into unit dosage forms such as tablets or capsules. This solid preformulation composition can then subdivided into unit dosage forms of the type described above.

The dosage forms of the present invention can be manufactured using processes that are well known to those of skill in the art. For example, for the manufacture of bi-layered tablets, the agents can be dispersed uniformly in one or more excipients, for example, using high shear granulation, low shear granulation, fluid bed granulation, or by blending for direct compression. Diluents or fillers can be used to increase the bulk of a tablet so that a practical size is provided for compression. Binders can impart cohesive qualities to a tablet formulation and can be used to help a tablet remain intact after compression. Disintegrants can facilitate tablet disintegration after administration. Stabilizers can inhibit or retard drug decomposition reactions, including oxidative reactions. Surfactants can also include and can be anionic, cationic, amphoteric, or nonionic. If desired, the tablets can also comprise nontoxic auxiliary substances such as pH buffering agents, preservatives, e.g., antioxidants, wetting or emulsifying agents, solubilizing agents, coating agents, flavoring agents, and the like.

Extended or controlled-release formulations can comprise one or more combination of excipients that slow the release of the agents by coating or temporarily bonding or decreasing their solubility of the active agents. Examples of these excipients include cellulose ethers such as hydroxypropyl methylcellulose, polyvinylacetate-based excipients, and polymers and copolymers based on methacrylates and methacrylic acid.

Embodiments of pharmaceutical compositions described herein may also be administered topically to the skin of a subject. The agent or effective ingredient can be mixed with a pharmaceutically acceptable carrier or a base which is suitable for topical application to skin to form a dermatological composition. Suitable examples of carrier or base include, but not limited to, water, glycols, alcohols, lotions, creams, gels, emulsions, and sprays. A dermatological composition comprising an analgesic agent can be integrated into a topical dressing, medicated tape, dermal patch absorbing gel and cleansing tissues. In one embodiment of the invention, the dermatological composition comprises hydrocodone or oxycodone, acetaminophen, and promethazine.

Embodiments of pharmaceutical compositions described herein may also be in liquid form. The liquid formulations can comprise, for example, an agent in water-in-solution and/or suspension form, and a vehicle comprising polyethoxylated castor oil, alcohol and/or a polyoxyethylated sorbitan mono-oleate with or without flavoring. Each dosage form comprises an effective amount of an active agent and can optionally comprise pharmaceutically inert agents, such as conventional excipients, vehicles, fillers, binders, disintegrants, solvents, solubilizing agents, sweeteners, coloring agents and any other inactive agents that can be included in pharmaceutical dosage forms for oral administration. Examples of such vehicles and additives can be found in Remington's Pharmaceutical Sciences, 17th edition (1985).

The compositions of the present invention can also be administered in injection-ready stable liquids for injection or I.V. drip. For example, saline or other injection-ready liquid can be mixed with an opioid analgesic (e g., hydrocodone or oxycodone), a non-opioid analgesic (e.g., acetaminophen) and an antihistamine (e.g., promethazine).

It will be appreciated that any number of administration methods known in the art may be used to administer the pharmaceutical compound containing active ingredients such as BSO. The administration method will determine the most effective dosage forms.

Embodiments of the present invention include the use of buthionine-sulfoximine in the manufacture of a pharmaceutical composition for treating a disease or disorder associated with high glucose levels in a subject

### Experiments and Results

Experimentation was done with Akita mice, which harbor a similar mutation in the proinsulin gene as human patients with MIDY, are used as a laboratory model for MIDY. Due to the genetic mutation, Akita mice are highly susceptible to proinsulin misfolding and exhibit severe disturbances in glucose homeostasis. The Akita strain is a monogenic model for phenotypes associated with type 1 diabetes. A spontaneous mutation in the insulin 2 gene leads to incorrect folding of the proinsulin protein producing toxicity in pancreatic beta-cells, reduced beta-cell mass and reduced insulin secretion. As a result, Akita mice suffer with glucose levels that are two to four-fold higher than healthy mice. Accordingly, experimental results show effects of embodiments of the present invention on glucose homeostasis, i.e., fasting glucose levels and glucose tolerance, and improper protein folding.

Animals: C57BL/6-Ins2^{Akita}/J (Akita) are an excellent model for Mutant-insulin Diabetes of Youth. Akita mice suffer from diabetes due to misfolded proinsulin, as the gene that codes for proinsulin (Ins2) is mutated. Two male heterozygous Akita mice and several C57BL/6-J (Wild-type) female mice, which do not harbor the mutation in Ins2 gene, were bought from Jackson laboratories (The Jackson Laboratory Inc., ME). One male Akita mice was bred to female mice and the progeny were back-crossed to obtain enough number of heterozygous and wild-type male mice. The genotype of the mice was confirmed by using methods described previously. Briefly, the polymerase chain reaction product of the amplicons from the mutated region of the Ins 2 gene were subjected to restriction enzyme digestion and the products of the digestion were separated in agarose gels. Based on the number of fragments that resulted from the digestion, the genotypes of mice were confirmed. Only heterozygous male mice were used in the experiments. After two rounds of breeding, 12 heterozygous Akita and 12 wild-type Akita mice. After reaching 15-weeks of age, mice in each genotype were randomized into two groups of 6 animals each (n=6/group). All mice were given either 0 mM or 15 mM of D,L-Buthionine-(S,R)-sulfoximine (BSO, Toronto Research Chemicals Inc., ON, CA) administered in drinking water. Fresh water and chow (Laboratory Rodent Diet 5001, PMI Nutrition International, Brentwood, MO) was provided every week with ad libitum access.

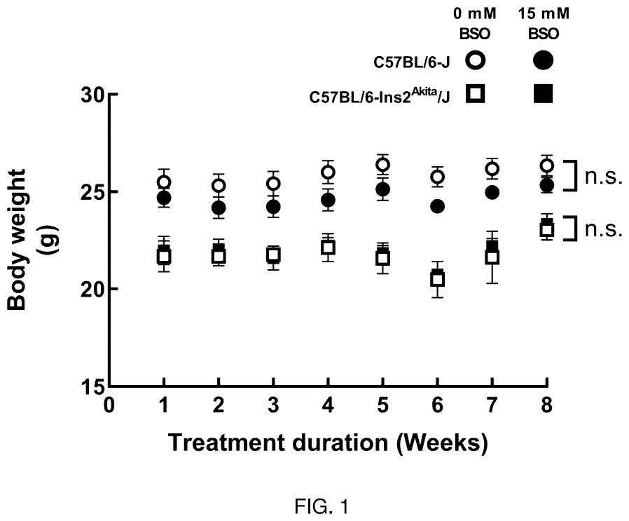

Experiment 1. Effect of D, L-Buthionine-(S, R)-sulfoximine (BSO) on body weight. Wild-type (C57BL/6-J, n=12) and Akita mice (C57BL/6-Ins2^{Akita}/J, n=12) obtained from in-house breeding were randomly divided into two groups of six each. Each group was offered water with or without 15 mM BSO continuously for eight weeks. Body weight was monitored every week. The results are shown in FIG. 1. A lack of the effect of BSO on body weight in either Akita mice of wild-type mice shows that BSO is not toxic.

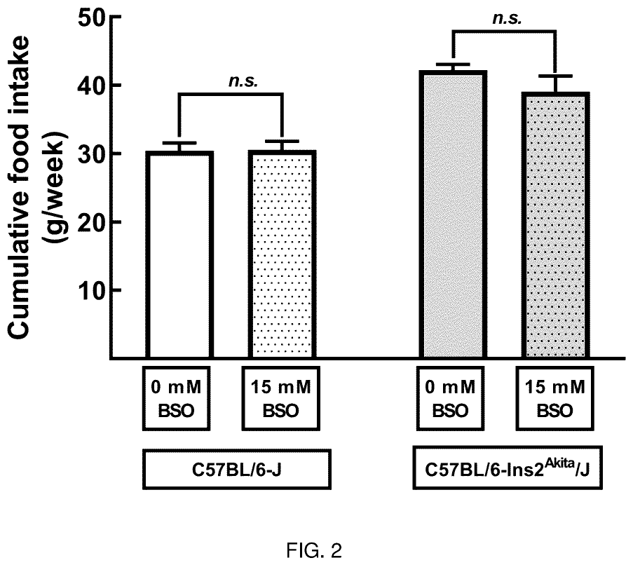

Experiment 2. Effect of D, L-Buthionine-(S, R)-sulfoximine (BSO) on food intake. Wild-type (C57BL/6-J, n=12) and Akita mice (C57BL/6-Ins2^{Akita}/J, n=12) obtained from in-house breeding were randomly divided into two groups of six each. Each group was offered water with or without 15 mM BSO continuously for eight weeks. All mice were given ad libitum access to (Laboratory Rodent Diet 5001, PMI Nutrition International, Brentwood, MO). Food intake was measured every week. The results are shown in FIG. 2. BSO does not affect the food intake in either the wild-type or in Akita mice.

Body weight (see FIG. 1) and food consumption (see FIG. 2) was monitored weekly. BSO treatment was continued for eight weeks. An Insulin Tolerance Test (ITT) was done during third week, and glucose tolerance test (GTT) was done during the fifth week after BSO treatment was initiated. Animals were sacrificed at the end of eight weeks on BSO treatment and blood and tissues were collected and immediately frozen at —80C.

Experiment 3. Effect of D, L-Buthionine-(S, R)-sulfoximine (BSO) on blood glutathione concentration. After eight weeks on 15 mM BSO, mice were sacrificed and the blood was collected for determining glutathione concentration. Glutathione was determined as described in experimental methods section. As expected BSO treatment decreased glutathione concentration in both wild-type and Akita mice.

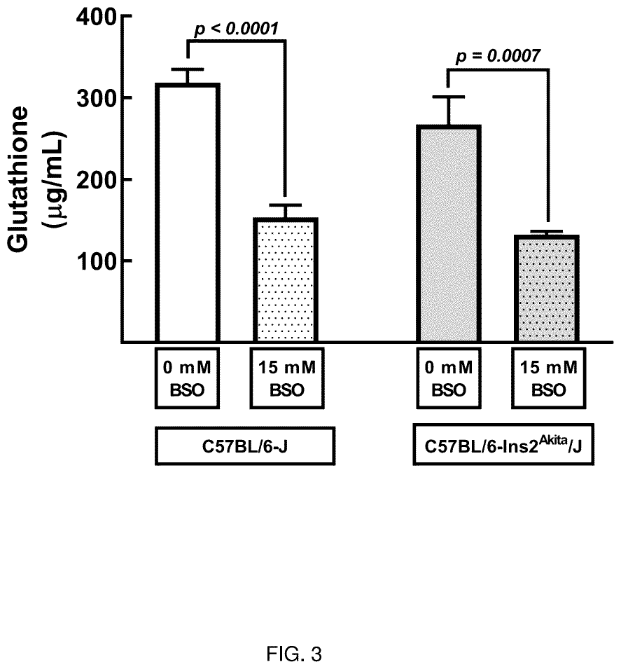

On the day of sacrifice, blood was collected from retro-orbital plexus. A 10 μL of blood was immediately deproteinized by adding to 90 μL of 5% metaphosphoric acid (Millipore-Sigma, St. Louis, MO). Deproteinized blood was immediately frozen and stored at −80 C. until the day of analysis. Glutathione was determined by a colorimetric method based on an enzymatic recycling method using 5,5′-dithiobis-(2-nitrobenzoic acid) as described earlier (Teitze et al). Using a standard curve with known concentrations the glutathione concentrations were determined and expressed as μg/mL blood. See FIG. 3.

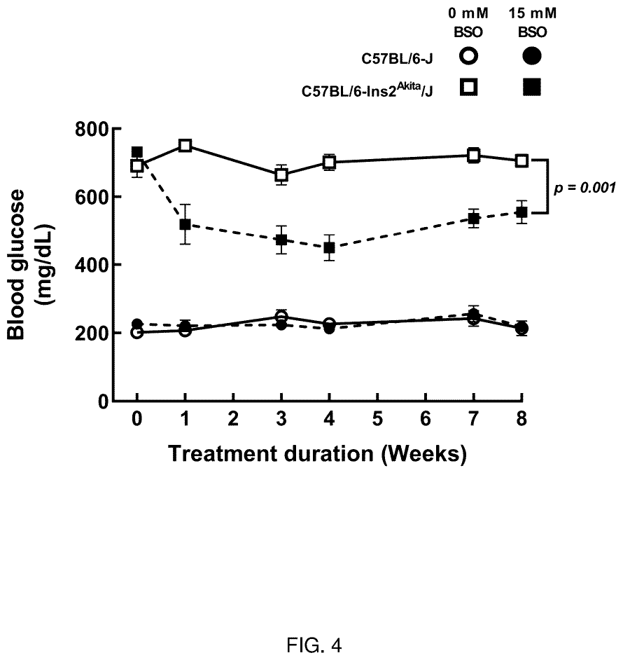

Experiment 4. Effect of D, L-Buthionine-(S, R)-sulfoximine (BSO) on fasting glucose. Fasting glucose was determined using AlphaTrak-2 glucometer. Mice were fasted for six hours before starting them on the BSO and 1, 3, 4, 7, and 8 weeks after treating with BSO. Decrease in glucose levels in Akita was evident within one week after treating with BSO and persisted throughout the study. Fasting (6 hr) blood glucose was determined from tail clips before starting the mice on BSO and 1, 3, 4, 7, and 8 weeks after treating with BSO. The results are shown in FIG. 4. AlphaTrak-2 (Parsipanny, NJ,) glucometer and compatible strips was used to determine blood glucose. As shown in FIG. 4, the glucometer has an upper detection limit of 750 mg/dL. Any measurement above the detection limit of the glucometer was considered as 751 mg/dL.

Experiment 5. Effect of D, L-Buthionine-(S, R)-sulfoximine (BSO) on glucose tolerance. Glucose tolerance test was conducted using AlphaTrak-2 glucometer. Mice were intraperitoneally injected with 0.85 g/Kg body weight D-glucose and blood glucose was determined at specified intervals. Despite the decrease in glutathione in wild type mice, BSO did affect the area under the curve of wild-type mice. But, BSO decreased the area under the curve of Akita mice to less than 50%. Findings show that the effect is specific to Akita mice, which harbor the mutant proinsulin gene.

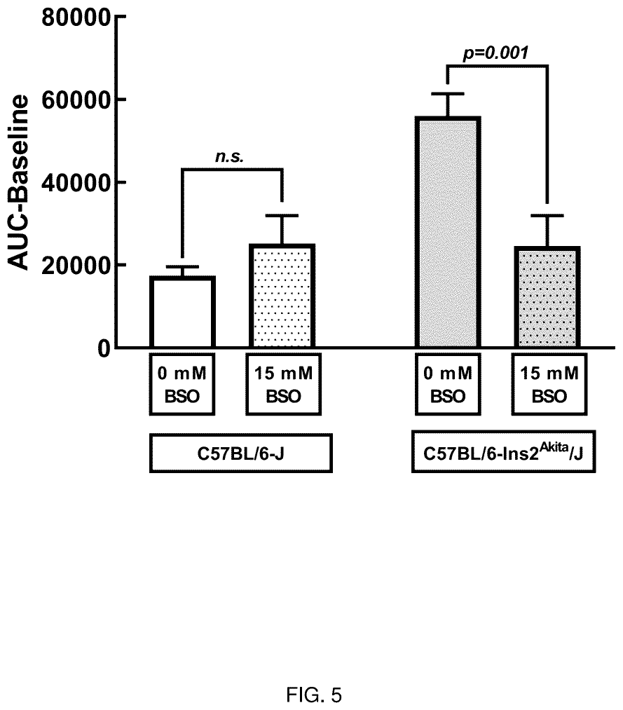

GTT was conducted after fasting the mice for 6 hours. All mice were injected with an intraperitoneal injection of D-glucose (Millipore-Sigma, St. Louis, MO) at a dose of 0.85 g/Kg body weight. Blood glucose was measured from tail clips using AlphaTrak-2 (Parsipanny, NJ,) glucometer and compatible strips. Blood glucose was measured at 0, 5, 15, 30, 60, 90, 120, 150, 180, and 210 min after injection. Area under the curve of the GTT was calculation using the trapezoid method and baseline values were subtracted. The cumulative area under the curve is represented in bar graphs as shown in FIG. 5.

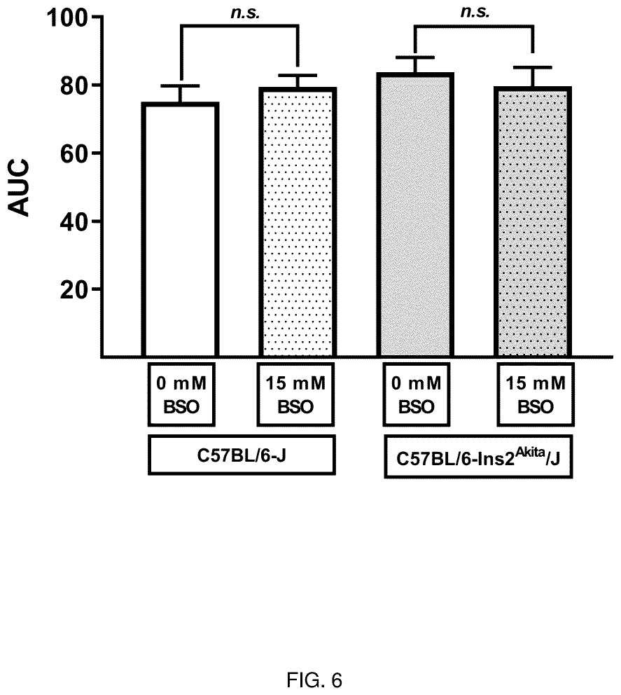

Experiment 6. Effect of D, L-Buthionine-(S, R)-sulfoximine (BSO) on insulin tolerance. Insulin tolerance test was conducted using AlphaTrak-2 glucometer. Mice were intraperitoneally injected with 1 U/Kg body weight of insulin and blood glucose was determined at specified intervals. No effect of BSO on area under the curve of either wild-type or Akita mice clearly indicates that the decrease in the blood glucose is not due to changes in insulin tolerance by peripheral tissues. ITT was conducted after fasting the mice for 6 hours. All mice were injected with an intraperitoneal injection of insulin (Humulin, Lilly Medical, Indianapolis, IN) at a dose of 1 U/Kg body weight. Blood glucose was measured from tail clips using AlphaTrak-2 (Parsipanny, NJ,) glucometer and compatible strips. Blood glucose was measured at 0, 5, 15, 30, 60, 90, 120, 150, 180, and 210 min after injection. The glucometer has an upper detection limit of 750 mg/dL. Any measurement above the detection limit of the glucometer was considered as 751 mg/dL. Area under the curve of the ITT was calculation using the trapezoid method and baseline values were subtracted. The cumulative area under the curve is represented in bar graphs as shown in FIG. 6.

It should be noted that the compositions, methods, and used discussed above are intended merely to be examples and should not be interpreted to limit the scope of the invention. Various embodiments may omit, substitute, or add various procedures or substances as appropriate. Various modifications and changes may be made as would be obvious to a person skilled in the art having the benefit of this disclosure. Also, features described with respect to certain embodiments may be combined in various other embodiments. Also, it should be emphasized that science evolves and the present invention is intended to embrace all such modifications, changes and evolutions. Thus, various modifications, alternative constructions, and equivalents may be used without departing from the spirit of the invention. The scope of the present invention is defined by the appended claims.

What is claimed:

1. A method of preventing or treating diseases and disorders associated with improper protein folding, the method comprising administering an effective amount of buthionine-sulfoximine to a subject to decrease improper protein misfolding in the subject's eukaryotic cells.

2. The method of claim 1, wherein an effective amount of buthionine-sulfoximine depletes glutathione by less than 50%.

3. The method of claim 1, wherein the buthionine-sulfoximine comprises at least one of L-buthionine-S-sulfoximine, L-buthionine-R-sulfoximine, D-buthionine-S-sulfoximine, D-buthionine-R-sulfoximine, a pharmaceutically-acceptable salt of any of the forgoing, or combinations thereof.

4. The method of claim 1, wherein an effective amount of buthionine-sulfoximine comprises at least about 0.01 g/Kg body weight.

5. The method of claim 4, wherein an effective amount of buthionine-sulfoximine comprises between about 0.1 g/Kg body weight and about 10 g/Kg body weight.

6. The method of claim 4, wherein an effective amount of buthionine-sulfoximine is an amount sufficient to increase an oxidation level within a eukaryotic cell without causing the cell to become toxic.

7. The method of claim 4, wherein administering an effective amount of a treatment compound into the subject comprises at least one of ingesting the treatment compound, injecting the treatment compound, applying the treatment compound topically or combinations thereof.

8. The method of claim 1, wherein the diseases and disorders associated with improper protein folding comprise diseases and disorders associated with high levels of glucose, and wherein administering an effective amount of an active ingredient comprising buthionine-sulfoximine to a subject reduces the subject's cellular glucose levels.

9. The method of claim 8, wherein administering an effective amount of the active ingredient reduces the subject's blood glucose levels by more than about 3%, 6%, 9%. 12%. 15%. 18%, 21%, 24%, 27%, or 30%.

10. The method of claim 8, wherein administering an effective amount of the active ingredient increases glucose tolerance by more than about 1%, 5%, 10%, 15%, 20%, 25%, 30%, 35%, 40%, 45%, or 50%.

11. The use of a composition comprising buthionine-sulfoximine in the manufacture of a pharmaceutical composition for treating a disease or disorder associated with improper protein folding.
